(19) United States
(12) Patent Application Publication
(10) Pub. No.: US 2024/0293336 A1
(43) Pub. Date: Sep. 5, 2024

(54) PERCUTANEOUS ABSORPTION FORMULATION FOR TREATING SLEEP DISORDERS

(71) Applicant: SINSIN PHARM CO., LTD., Gyeonggi-do (KR)

(72) Inventors: Jung Sik LEE, Gyeonggi-do (KR); Sun Ho Lee, Gyeonggi-do (KR); Mun Seok Han, Gyeonggi-do (KR); Woo Young Lee, Gyeonggi-do (KR); Sang Lin Kim, Seoul (KR); Han Ki Kim, Gyeonggi-do (KR)

(21) Appl. No.: 18/646,212

(22) Filed: Apr. 25, 2024

Related U.S. Application Data
(63) Continuation of application No. 16/345,948, filed on Apr. 29, 2019, now abandoned.

(30) Foreign Application Priority Data
Oct. 31, 2016 (KR) 10-2016-0143368
Oct. 25, 2017 (KR) 10-2017-0138990

(51) Int. Cl. A61K 9/70; A61K 31/4045; A61K 47/10; A61K 47/12; A61K 47/14; A61K 47/22; A61K 47/26; A61K 47/32; A61K 47/34; A61K 47/38

(52) U.S. Cl. CPC A61K 9/7061; A61K 9/7053; A61K 31/4045; A61K 47/10; A61K 47/12; A61K 47/14; A61K 47/22; A61K 47/26; A61K 47/32; A61K 47/34; A61K 47/38

(57) ABSTRACT

Disclosed is a percutaneous absorption formulation containing melatonin which is a pharmacologically active substance useful for treating insomniac patients and sleep disorder patients.

### FIELD

The present invention relates to a formulation of percutaneous absorption of melatonin, which contains, melatonin as an active ingredient, to improve patient's medication adherence, enabling the drug to be stably maintained without forming a crystal, albeit its high concentration in an adhesive matrix, and can deliver the active ingredient into the body through the skin.

### BACKGROUND

Melatonin is a hormone secreted by the mammalian pineal gland. The circadian rhythm of melatonin secretion is photoperiodically regulated. The concentration of melatonin in plasma begins to increase at around 9:00 pm, reaches its peak from 2:00 am to 4:00 am, and returns back to its original level from 7:00 am to 9:00 am.

Melatonin has a sleep-inducing effect, and thus is used for treatment of insomniac patients and sleep disorder patients. However, melatonin, when administered orally, undergoes serious liver first-pass, has low bioavailability due to its short half-life (about 1 hour), and cause huge changes in blood concentration. Thus, it has been reported that the bioavailability of melatonin is only 20%.

Therefore, for effective treatment with melatonin, there is a need to develop a novel formulation which can avoid liver first-pass, can maintain the blood melatonin concentration at a constant level over a long period of time, and can be absorbed rapidly.

### SUMMARY

### Technical Problem

A reservoir-type percutaneous absorption formulation comprises a gel-type drug-containing reservoir layer, a release layer for controlling drug release, and an adhesive material for attaching to the skin. However, there are problems such as, that the reservoir may be damaged during storage or use, and that if the reservoir is damaged during use by a patient, the drug may be absorbed at a level higher than its predetermined level, resulting in patient risk.

In addition, when an acrylic polymer is synthesized directly during the preparation of a monolithic-type formulation, a problem arises in that the toxicity and stability of an adhesive as an adhesive agent are not verified. This is risky and inefficient because percutaneous absorption formulation manufacturers generally produce percutaneous absorption formulations using commercialized adhesives.

Therefore, there is a need to develop a formulation for percutaneous absorption of melatonin, which can overcome the above-described disadvantages of a percutaneous absorption formulation, capable of controlling drug release, has high stability, convenient to store and use, and easy to manufacture.

This present invention is intended to provide a monolithic thin film-type formulation for percutaneous absorption of melatonin, which comprises a combination of raw materials with verified safety. Thus, it has high safety and is convenient to store and use. Unlike the conventional reservoir-type formulation, the monolithic percutaneous absorption formulation can store, stabilize, and control percutaneous absorption of drug.

### Technical Solution

To solve the above problem, without taking a reservoir type, the present inventors have combined a suitable adhesive material with raw materials thereby completing a monolithic thin film-type formulation for percutaneous absorption of melatonin, which has improved drug stability and possible for drug to be delivered at a constant level through the skin.

### Advantageous Effects

The formulation for percutaneous absorption of melatonin according to the present invention enables controlled drug release, has high stability, is convenient to store and use, and is easy to manufacture. In addition, the percutaneous absorption formulation, according to the present invention, can significantly improve patient's medication compliance compared to an oral formulation, and at the same time, can deliver an effective amount of necessary drug to a patient, indicating that it can be advantageously used for the treatment of insomnia and sleep disorders. In addition, the amount of the formulation remaining in the skin after removal can be minimized.

### BRIEF DESCRIPTION OF DRAWINGS

FIG. 1 is a sectional view of a monolithic percutaneous absorption formulation manufactured by the present invention. A is a supporting layer, B is a drug-containing adhesive layer, and C is a releasing layer.

FIG. 2 is a graph showing cumulative percutaneous absorption for Examples 18 to 23 of the present invention and Comparative Examples 3 and 4.

FIG. 3 is a graph showing cumulative percutaneous absorption for Examples 24 to 29 of the present invention.

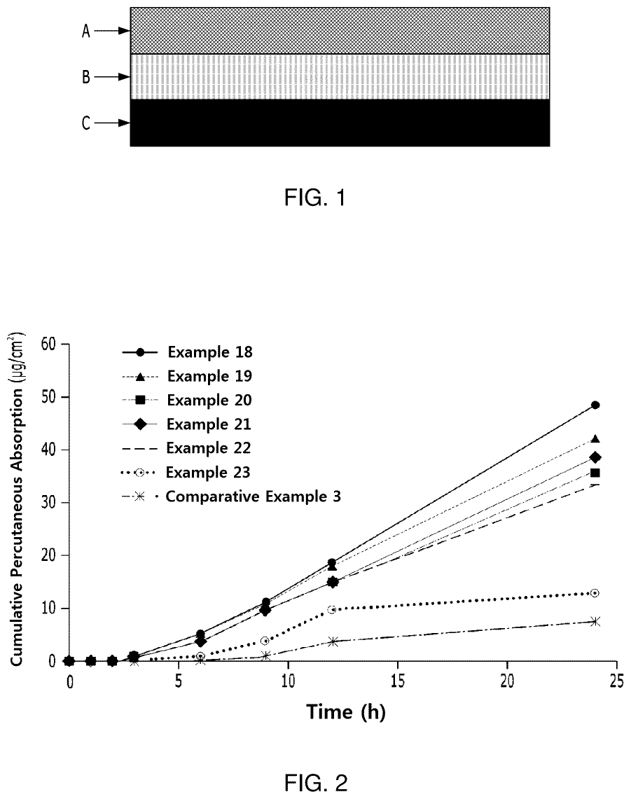

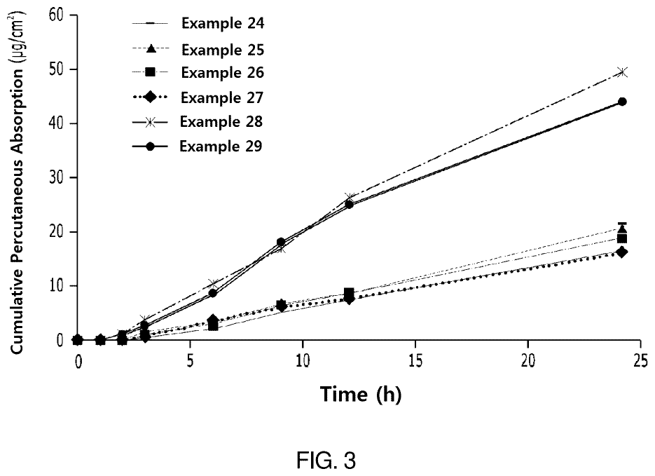

### BEST MODE

The present invention relates to a percutaneous absorption formulation comprising melatonin as an active ingredient, specifically to a monolithic formulation for percutaneous absorption of melatonin.

The percutaneous absorption formulation of the present invention comprises: (a) a drug-containing adhesive layer which contains melatonin or a pharmaceutically acceptable salt as an active ingredient, and a polymeric adhesive agent; (b) a supporting layer; and (c) a releasing layer. It may be used for the treatment of insomnia and sleep disorders.

In the present invention, the drug-containing adhesive layer may contain, in addition to melatonin, pharmaceutically acceptable salt, a polymeric adhesive agent, a solubilizing agent, a crystallization inhibitor, a percutaneous absorption enhancer, and an antioxidant.

As the polymeric adhesive agent that is used in the present invention, any pressure-sensitive adhesive may be used without limitation, but an acrylic adhesive is more preferable.

An acrylic adhesive that may be used in the present invention is an acrylic polymer adhesive agent composed of either acrylate or a copolymer of acrylate and vinyl acetate. The acrylic polymer adhesive agent may be one or two or more selected from the group consisting of (i) one having no functional group, (ii) one having a hydroxyl (—OH) group as a functional group, (iii) one having a carboxyl (—COOH) group as a functional group, and (iv) one having both a hydroxyl group and a carboxyl group as functional groups. Preferably, an acrylic adhesive having a carboxyl (—COOH) group or containing both a carboxyl group and a hydroxyl (—OH) group may be used.

Since melatonin has a moiety having non-covalent electrons in its structure, an adhesive having a hydroxyl group rich in electrons is advantageous for percutaneous absorption, but may have a problem in that it easily forms crystals. A non-functional acrylic adhesive may have a problem in that the adhesive easily forms crystals, because the solubility of the adhesive itself is lowered due to the functional group of melatonin.

On the other hand, an adhesive having a carboxylic group can solubilize the drug by interaction with the functional group of melatonin, and crystallization of the active ingredient component can be effectively inhibited by the use of little amount of a solubilizing agent and a crystallization inhibitor.

In addition, the polymeric adhesive agent that is used in the present invention may be an adhesive agent comprising a hydrophobic polymer. As a hydrophobic polymer, one or two or more can be selected from the group consisting of polyisoprene, polyisobutylene, polybutadiene, a polystyrene-butadiene copolymer, a polystyrene-isoprene copolymer, a styrene-isoprene-styrene block copolymer, a styrene-butadiene-styrene block copolymer, butyl rubber, natural rubber, an ethylene-vinyl acetate copolymer, polysiloxane, and methacrylic acid-based polymers.

The polymeric adhesive agent may further comprise a tackifying resin and a plasticizer. In this case, the hydrophobic polymer may be contained in an amount of 20 to 60 wt %, the tackifying resin may be contained in an amount of 20 to 50 wt %, and the plasticizer may be contained in an amount of 2 to 30 wt %.

An acrylic adhesive is preferable in terms of solubility of drug or adhesion.

In the present invention, a solubilizing agent may be used for stable solubilization of melatonin in percutaneous absorption formulations. As a solubilizing agent, a pyrrolidone derivative, glycol, propylene carbonate, ether, or polyoxyethylene fatty acid ester may be used. Preferably, one or two or more solubilizing agent can be selected from the group consisting of N-methylpyrrolidone, dipropylene glycol, propylene glycol, propylene carbonate, ethoxydiglycol, diethylene glycol monoethyl ether, triacetin, triethyl citrate, trolamine, tromethamine, bis-Tris, aminomethyl propanediol, aminoethyl propanediol, polyoxyethylene sorbitan monooleate, and PEG-8 caprylic/capric glycerides.

Melatonin is highly soluble in substances having both hydrophilic and hydrophobic properties, due to the molecular characteristics of melatonin having both hydrophilic and hydrophobic moieties. The solubilizing agent of the present invention is used in an amount of 1 to 30 wt %, preferably 1 to 15 wt %, based on the total weight of the drug-containing adhesive layer. If the solubilizing agent is used in an amount larger than 30 wt %, it may damage the user's skin, and may also reduce the physical strength of the percutaneous absorption formulation.

In the present invention, a crystallization inhibitor of the drug may be contained in order to prevent the crystallization of melatonin in the percutaneous absorption formulation. The crystallization inhibitor may be selected one or two or more from the group consisting of polyvinylpyrrolidone, a methacrylic copolymer, an amino acrylic methacrylate copolymer, a butyl methacrylic methacrylate copolymer, and hydroxypropyl cellulose. Preferably, it comprises one or two or more of an amino acrylic methacrylate copolymer and polyvinylpyrrolidone.

The crystallization inhibitor may be used in an amount of 0.05 to 5 wt %, preferably 0.05 to 2.5 wt %, based on the total weight of the drug-containing adhesive layer. The crystallization inhibitor is a polymer, and hence if it is used in an amount larger than 5 wt %, problems such as sticking with an acrylic adhesive may occur, and adhesive strength may also be reduced.

In the present invention, the percutaneous absorption formulation may further comprise a percutaneous absorption enhancer. As the percutaneous absorption enhancer, fatty acid ester, a nonionic surfactant, a pyrrolidone derivative, fatty acid, fatty acid alcohol, or fatty acid ester may be used. Preferably, fatty acid ester is used.

A C₈₋₁₈ aliphatic derivative that may be used as the percutaneous absorption enhancer in the present invention may be one or two or more selected from the group consisting of glycerol lauryl alcohol, oleyl alcohol, myristic acid, isopropyl myristate, sorbitan monooleate, propylene glycol monolaurate, propylene glycol monooleate, oleoyl macrogolglycerides, oleic acid, lauroyl macrogol glyceride, linolenic acid, linoleoyl macrogol glyceride, propylene glycol dicaprylate/caprate, sorbitan monostearate, sorbitan monooleate, glycerol monooleate, glycerol monolaurate, propylene glycol monolaurate, propylene glycol monocaprylate, sorbitan monolaurate, lauryl lactate, PEG-8 caprylic/capric triglycerides, polyoxyethylene sorbitan monolaurate, corn oil PEG-8 esters, and corn oil PEG-6 esters.

More preferably, as the C₈₋₁₈ aliphatic derivative, one or two or more can be selected from the group consisting of glycerol monooleate, glycerol monolaurate, propylene glycol monolaurate, sorbitan monooleate, sorbitan monolaurate, corn oil PEG-8 esters, and corn oil PEG-6 esters.

The percutaneous absorption enhancer of the present invention may be used in an amount of 1 to 30 wt %, preferably 1 to 15 wt %, based on the total weight of the drug-containing adhesive layer. If the percutaneous absorption enhancer is used in an amount larger than 30 wt %, then it will no longer improve the percutaneous absorption of the drug, resulting in a decrease in the physical strength of the percutaneous absorption formulation, and in some cases, can cause skin troubles in the user.

The percutaneous absorption formulation of the present invention may further comprise an antioxidant in order to inhibit degradation and denaturation in the percutaneous absorption formulation. Since melatonin also acts as an oxygen radical scavenger in vivo, the percutaneous absorption formulation may contain an antioxidant or the like to suppress oxidation.

As the antioxidant, one or two or more can be selected from the group consisting of butyl hydroxy toluene, butyl hydroxy anisole, propyl galate, ascorbic acid, tocopherol, tocopherol acetate, and ascorbyl palmitate may be used. Preferably, butyl hydroxy toluene, tocopherol, tocopherol acetate, or butyl hydroxy anisole.

The antioxidant may be used in an amount of 0.1 to 5 wt %, preferably 0.1 to 1 wt %, based on the total weight of the drug-containing adhesive layer. If the antioxidant is used in an amount larger than 5 wt %, the antioxidant effect is no longer improved.

In the present invention, the drug-containing adhesive layer may contain melatonin or a pharmaceutically acceptable salt thereof in an amount of 3 to 20 wt % based on the total weight of the drug-containing adhesive layer. Meanwhile, the drug-containing adhesive layer may further contain, in addition to melatonin or a pharmaceutically acceptable salt thereof, a solubilizing agent, a crystallization inhibitor, a percutaneous absorption enhancer and an antioxidant in the above-described amounts, and may further contain a suitable amount (50 to 94.85 wt %) of a polymeric adhesive agent.

In the present invention, the supporting layer is used to prevent melatonin from being lost during attachment to the skin or storage, and is made of a thin, flexible material, that causes no skin allergic reaction. The supporting layer may be made of a material, such as polyethylene terephthalate (PET), polyethylene (PE), polypropylene (PP), ethylene-vinyl acetate (EVA) copolymer, nylon, or the like. A film made of this material may be used alone, or in a laminated film form obtained by laminating two or more films. In addition, when two or more films are laminated, a film having aluminum deposited may be used for light shielding and prevention of moisture penetration. Furthermore, in order to facilitate patch formation, nonwoven fabric, cotton cloth, fabric or the like may be laminated on to this film, or the above-mentioned fabric may also be used alone.

In the present invention, the releasing layer is not particularly limited, as long as it protects the drug-containing product during packaging or storage of the product and provides convenience so that it can be easily removed when using the product. It may be either a film produced from polyester, polyvinyl chloride, polyvinylidene chloride, polyethylene terephthalate, polyethylene, ethylene vinyl acetate or the like, or a film obtained by laminating polyolefin on paper such as wood-free paper or glassine paper. The adhesive layer-contacting surface of the releasing film is coated with silicone resin or fluorine resin. As the release layer, a polyethylene terephthalate film showing excellent long-term stability of the drug is preferably used.

### DETAILED DESCRIPTION

### EXAMPLES

Hereinafter, the present invention will be described in detail with reference to examples. However, the following examples are only illustrative of the present invention, and the scope of the present invention is not limited to the following examples.

### 1. Examples 1 to 5 and Comparative Examples 1 to 2

10 wt % melatonin was placed in a 70 mL vial and dissolved in methanol, and then 90 wt % adhesive was added thereto, followed by mixing in a roll mixer for 2 hours. Next, the mixture was allowed to stand for 30 minutes to remove bubbles, after which a silicone-treated polyethylene terephthalate release film was placed on a coating machine, and the mixed adhesive was dried, and then coated onto the release film to 70 μm. Thereafter, the resultant was dried in an oven at 80° C. for 3 minutes, and then a supporting layer film was laminated thereon and compressed using a roller press. The resultant was cut of a patch cutter, thereby manufacturing a patch having an area of 10 cm². The contents of the components according to each Example are shown in Table 1 below. Next, the physical properties of the patch and crystal formation were observed, and a percutaneous absorption test was performed. The observation and test results are also shown in Table 1 below.

### Percutaneous Absorption Test

Phosphate buffered saline solution (PBS solution, pH=7.4) as a receptor was placed in a Franz-type diffusion cell and stirred with a magnetic stirrer at a constant stirring speed of 600 rpm while maintaining 32° C. which is similar to skin temperature, thereby removing dissolved gas from the solution. Next, the patch cut in accordance with the upper donor cell of the Franz-type diffusion cell was attached to a membrane for percutaneous absorption (Strat-M, Merck KGaA, Darmstadt, Germany) and mounted in the Franz-type diffusion cell. Next, the solution in the receptor compartment was sampled at predetermined time intervals, and a fresh buffer solution was supplemented in an amount equal to the sampled amount. The sample was analyzed by high-performance liquid chromatography (HPLC) under the following conditions:

### Analysis Conditions

Column: C18, 4.6 mm, 5 μm (Agilent eclips SB-18);
Mobile phase: Methanol:water=35:65 (v/v);
Flow rate: 1 mL/min;
Column temperature: 35° C.;
Amount injected: 20 μl;
Detection: UV 229 nm.

TABLE 1

| | | | | | | | Comparative | |
|---|---|---|---|---|---|---|---|---|
| | | Examples (wt %) | | | | | Examples (wt %) | |
| Classification | | 1 | 2 | 3 | 4 | 5 | 1 | 2 |
| Melatonin | | 10 | 10 | 10 | 10 | 10 | 10 | 10 |
| Adhesive | Duro- | 90 | | | | | | |
| | Tak ™ | | | | | | | |
| | 87-9301 | | | | | | | |
| | Duro- | | 90 | | | | | |
| | Tak ™ | | | | | | | |
| | 87-2516 | | | | | | | |
| | Duro- | | | 90 | | | | |
| | Tak ™ | | | | | | | |
| | 87-2074 | | | | | | | |
| | Duro- | | | | 90 | | | |
| | Tak ™ | | | | | | | |
| | 87-4098 | | | | | | | |
| | Duro- | | | | | 90 | | |
| | Tak ™ | | | | | | | |
| | 87-2852 | | | | | | | |
| | Duro- | | | | | | 90 | |
| | Tak ™ | | | | | | | |
| | 87-608A | | | | | | | |
| | (PIB) | | | | | | | |
| | Sanicare | | | | | | | 90 |
| | HM8663 | | | | | | | |
| | (SIS) | | | | | | | |
| Patch | Initial | Colorless | Yellow | Colorless | White | Colorless | Colorless | Colorless |
| appearance | | translucent | translucent | transparent | translucent | transparent | transparent | transparent |
| | After 1 | Yellow | Yellow | Yellow | Yellow | Yellow | Yellow | Yellow |
| | month | translucent | translucent | transparent | translucent | transparent | transparent | transparent |
| Physical properties and | | Good | Good | Good | Good | Good | Good | Good |
| adhesive strength | | | | | | | | |
| Percutaneous absorption | | 0.37 | 1.01 | 0.44 | 0.4 | 0.23 | 1.12 | 0.89 |
| (μg/cm²/24 h) | | | | | | | | |
| Crystal formation (1 | Crystal | Crystal | None | Crystal | None | Crystal | Crystal | |
| month) | precipitation | precipitation | | precipitation | | precipitation | precipitation | |

Adhesive agent used

| Name of raw material | Kind | Functional group |
|---|---|---|
| Duro-Tak ™ 87-9301 | Acrylic polymer adhesive agent | Non |
| Duro-Tak ™ 87-2516 | Acrylic polymer adhesive agent | —OH |
| Duro-Tak ™ 87-2074 | Acrylic polymer adhesive agent | —COOH/—OH |
| Duro-Tak ™ 87-4098 | Acryl-vinyl acetate-based polymeric adhesive agent | Non |
| Duro-Tak ™ 87-2852 | Acrylic polymer adhesive agent | —COOH |
| Duro-Tak ™ 87-608A | Polyisobutylene adhesive agent | N/A |
| Sanicare HM 8663 | Styrene-isoprene-styrene copolymer adhesive agent | N/A |

Duro-Tak™ 87-2516 was the adhesive showing the highest percutaneous absorption, but formed a crystalline precipitate. The adhesives, which formed no crystalline precipitate, were Duro-Tak™ 87-2074 and Duro-Tak™ 87-2852, and Duro-Tak™ 87-2074 showed higher percutaneous absorption than Duro-Tak™ 87-2852. The rubber-based adhesives of the Comparative Examples showed a good percutaneous absorption, but formed a crystalline precipitate.

### Examples 6 to 10

Melatonin and a solubilizing agent were placed in a 70 mL vial and dissolved in methanol, and then an adhesive was added thereto, followed by mixing in a roll mixer for 2 hours. Next, the mixture was allowed to stand for 30 minutes to remove bubbles, after which a silicone-treated polyethylene terephthalate release film was placed on a coating machine, and the mixed adhesive was dried, and then coated on the release film to 70 μm. Thereafter, the resultant was dried in an oven at 80° C. for 3 minutes, and then a support layer film was laminated thereon and compressed using a roller press. The resultant was cut by using a patch cutter, thereby manufacturing a patch having an area of 10 cm². The contents of the components according to each Example are shown in Table 2 below. Next, the physical properties of the patch and crystal formation were observed. The observation and test results are also shown in Table 2 below.

TABLE 2

| | | Examples (wt %) | | | | |
|---|---|---|---|---|---|---|
| Classification | | 6 | 7 | 8 | 9 | 10 |
| Melatonin | | 10 | 10 | 10 | 10 | 10 |
| Adhesive | Duro-Tak ™ | 85 | 85 | 85 | 85 | 85 |
| | 87-2074 | | | | | |
| Solubilizing | NMP | 5 | | | | |
| agent | Transcutol CG | | 5 | | | |
| | Transcutol P | | | 5 | | |
| | Labrasol | | | | 5 | |
| | DPG | | | | | 5 |
| Patch | Initial | Colorless | Yellow | Colorless | White | Colorless |
| appearance | | translucent | translucent | transparent | translucent | transparent |
| | After 1 month | Yellow | Yellow | Yellow | Yellow | Yellow |
| | | translucent | translucent | transparent | translucent | transparent |

| | | | | | |
|---|---|---|---|---|---|
| Physical properties and | Good | Good | Good | Good | Good |
| adhesive strength | | | | | |
| Crystal formation (after 1 month) | Fine crystal | Fine crystal | Crystal | Crystal | Fine crystal |
| (microscopic observation) | formation | formation | formation | formation | formation |

NMP: N-methyl pyrrolidone;
Transcutol CG: ethoxydiglycol;
Transcutol P: diethylene glycol monoethyl ether;
Labrasol: PEG-8 caprylic/capric glyceride;
DPG: dipropylene glycol

When the manufactured patches were observed, N-methyl pyrrolidone, Transcutol CG and dipropylene glycol showed no crystal formation in visual observation, but showed fine crystal formation in microscopic observation, and yellowing in color in the manufactured patches has appeared. Thus, in order to effectively inhibit crystal formation in a patch, an additional study on a crystallization inhibitor was conducted, and an antioxidant formulation study was conducted to prevent yellowing.

### Examples 11 to 17

A crystallization inhibitor was placed and dissolved in a 70 mL vial, and then a solubilizing agent, an antioxidant and melatonin were added and dissolved, after which an adhesive was added thereto, followed by mixing in a roll mixer for 2 hours. Next, the mixture was allowed to stand for 30 minutes to remove bubbles, after which a silicone-treated polyethylene terephthalate release film was placed on a coating machine, and the mixed adhesive was dried, and then coated on the release film to 70 μm. Thereafter, the resultant was dried in an oven at 80° C. for 3 minutes, and then a support layer film was laminated thereon and compressed using a roller press. The resultant was cut by using a patch cutter, thereby manufacturing a patch having an area of 10 cm². The contents of the components according to each Example are shown in Table 3 below. Next, the physical properties of the patch and crystal formation were observed. The observation and test results are also shown in Table 3 below.

TABLE 3

| | | Examples (wt %) | | | | | | |
|---|---|---|---|---|---|---|---|---|
| Classification | | 11 | 12 | 13 | 14 | 15 | 16 | 17 |
| Melatonin | | 10 | 10 | 10 | 10 | 10 | 10 | 10 |
| Adhesive | Duro- | 83 | 83 | 83 | 83 | 83 | 83 | 83 |
| | Tak ™ | | | | | | | |
| | 87-2074 | | | | | | | |
| Solubilizing | DPG | 5 | 5 | 5 | 5 | 5 | | 5 |
| agent | | | | | | | | |
| Antioxidant | BHT | 0.5 | | 0.5 | 0.5 | 0.5 | 0.5 | 0.5 |
| | TA | | 05 | | | | | |
| Crystallization | PVP | 1.5 | 1.5 | | | | | |
| inhibitor | Eudragit | | | 1.5 | | | | |
| | L-100 | | | | | | | |
| | Eudragit | | | | 1.5 | | | |
| | S-100 | | | | | | | |
| | Plastoid B | | | | | 1.5 | | |
| | Eudragit | | | | | | 1.5 | |
| | E-100 | | | | | | | |
| | Klucel-LF | | | | | | | 1.5 |
| Patch | Initial | Colorless | Colorless | Colorless | Colorless | Colorless | Colorless | Colorless |
| appearance | | transparent | transparent | transparent | transparent | transparent | transparent | transparent |
| | After 1 | Colorless | Colorless | Colorless | Colorless | Colorless | Colorless | Colorless |
| | month | transparent | transparent | transparent | transparent | transparent | transparent | transparent |

| | | | | | | | |
|---|---|---|---|---|---|---|---|
| Physical properties and | Good | Good | Good | Good | Good | Good | Good |
| adhesive strength | | | | | | | |
| Crystal formation | None | None | Fine crystal | Fine crystal | Fine crystal | None | Crystal |
| (after 1 month) | | | formation | formation | formation | | formation |
| (microscopic observation) | | | | | | | |

DPG: dipropylene glycol;
BHT: butyl hydroxyl toluene;
TA: tocopherol acetate;
PVP: Polyvinyl Pyrrolidone;
Eudragit RL-100: Ammonio Methacrylate Copolymer, Type A
Eudragit RS-100: Ammonio Methacrylate Copolymer, Type B
Plastoid B: Butyl methacrylic methacrylate copolymer;
Eudragit E-100: Amino Methacrylate Copolymer;
Klucel-LF: Hydroxypropyl cellulose

In the test results, the antioxidant inhibited the oxidation of the active ingredient component, regardless of the class of the antioxidant component, and thus yellowing in the drug layer of each patch did not appear. In addition, polyvinyl pyrrolidone (PVP) and the amino methacrylate copolymer (Eudragit E-100) showed the highest inhibition of crystallization of the active ingredient component.

### Examples 18 to 23 and Comparative Example 3

A crystallization inhibitor was placed and dissolved in a 70 mL vial, and then a solubilizing agent, an antioxidant, a percutaneous absorption enhancer and melatonin were added thereto and dissolved, after which an adhesive was added thereto, followed by mixing in a roll mixer for 2 hours. Next, the mixture was allowed to stand for 30 minutes to remove bubbles, after which a silicone-treated polyethylene terephthalate release film was placed on a coating machine, and the mixed adhesive was dried, and then coated on the release film to 70 μm. Thereafter, the resultant was dried in an oven at 80° C. for 3 minutes, and then a support layer film was laminated thereon and compressed using a roller press. The resultant was cut by means of a patch cutter, thereby manufacturing a patch having an area of 10 cm². The contents of the components according to each Example are shown in Table 4 below. Next, the physical properties of the patch and crystal formation were observed, and a percutaneous absorption test was performed. The observation and test results are also shown in Table 4 below and FIG. 2.

TABLE 4

| | | | | | | | | Comparative |
|---|---|---|---|---|---|---|---|---|
| | | Examples (wt %) | | | | | | Example (wt %) |
| Classification | | 18 | 19 | 20 | 21 | 22 | 23 | 3 |
| Melatonin | | 10 | 10 | 10 | 10 | 10 | 10 | 10 |
| Adhesive | Duro- | 78 | 78 | 78 | 78 | 80.5 | 83 | 83 |
| | Tak ™ | | | | | | | |
| | 87-2074 | | | | | | | |
| Solubilizing | DPG | 5 | 5 | 5 | 5 | 5 | 5 | 5 |
| agent | | | | | | | | |
| Antioxidant | TA | 0.5 | 0.5 | 0.5 | 0.5 | 0.5 | 0.5 | 0.5 |
| Crystallization | PVP | 1.5 | 1.5 | 1.5 | 1.5 | 1.5 | 1.5 | 1.5 |
| inhibitor | | | | | | | | |
| Percutaneous | GML | 5 | | | | 2.5 | 5 | |
| absorption | GMO | | 5 | | | | | |
| enhancer | SMO | | | 5 | | | | |
| | GMC | | | | 5 | | | |
| Percutaneous absorption | | 47.82 | 42.15 | 34.23 | 37.95 | 33.27 | 13 | 7.4 |
| (μg/cm²/24 h) | | | | | | | | |
| Patch | Initial | Colorless | Colorless | Colorless | Colorless | Colorless | Colorless | Colorless |
| appearance | | transparent | transparent | transparent | transparent | transparent | transparent | transparent |
| | After 1 | Colorless | Colorless | Colorless | Colorless | Colorless | Colorless | Colorless |
| | month | transparent | transparent | transparent | transparent | transparent | transparent | transparent |

| | | | | | | | |
|---|---|---|---|---|---|---|---|
| Physical properties and | Good | Good | Good | Good | Good | Good | Good |
| adhesive strength | | | | | | | |
| Crystal formation | None | None | None | None | None | None | None |
| (after 1 month) | | | | | | | |
| (microscopic observation) | | | | | | | |

DPG: dipropylene glycol;
TA: tocopherol acetate;
PVP: polyvinyl pyrrolidone;
GML: glycerol monolaurate;
GMO: glycerol monooleate;
SMO: sorbitan monooleate;
GMC: glycerol monocaprylate

As the test results shown in Table 4 above, the manufactured patches effectively achieved the percutaneous absorption of melatonin.

It shows that Examples 18 to 22 showed an increase in percutaneous absorption due to the use of the percutaneous absorption enhancer, compared to Comparative Example 4. In addition, it shows that Examples 18 to 22 showed changes in percutaneous absorption depending on the kind of additive (such as percutaneous absorption enhancer) and the amount of additive used.

### Examples 24 to 29

A crystallization inhibitor was placed and dissolved in a 70 mL vial, and then a solubilizing agent, an antioxidant, a percutaneous absorption enhancer and melatonin were added thereto and dissolved, after which an adhesive was added thereto, followed by mixing in a roll mixer for 2 hours. Next, the mixture was allowed to stand for 30 minutes to remove bubbles, after which a silicone-treated polyethylene terephthalate release film was placed on a coating machine, and the mixed adhesive was dried, and then coated on the release film to 70 μm. Thereafter, the resultant was dried in an oven at 80° C. for 3 minutes, and then a support layer film was laminated thereon and compressed using a roller press. The resultant was cut by means of a patch cutter, thereby manufacturing a patch having an area of 10 cm². The contents of the components according to each Example are shown in Table 5 below. Next, the physical properties of the patch and crystal formation were observed, and a percutaneous absorption test was performed. The observation and test results are also shown in Table 5 below.

TABLE 5

| | | Examples (wt %) | | | | | |
|---|---|---|---|---|---|---|---|
| Classification | | 24 | 25 | 26 | 27 | 28 | 29 |
| Melatonin | | 10 | 10 | 10 | 10 | 10 | 10 |
| Adhesive | Duro- | 78 | 78 | 78 | 78 | 73 | 73 |
| | Tak ™ | | | | | | |
| | 87-2074 | | | | | | |
| Solubilizing | DPG | 5 | 5 | 5 | 5 | 5 | 5 |
| agent | | | | | | | |
| Antioxidant | TA | 0.5 | 0.5 | 0.5 | 0.5 | 0.5 | 0.5 |
| Crystallization | PVP | 1.5 | 1.5 | 1.5 | 1.5 | 1.5 | 1.5 |
| inhibitor | | | | | | | |
| Percutaneous | LNA | 5 | | | | | |
| absorption | MYA | | 5 | | | 10 | |
| enhancer | OLE | | | 5 | | | 10 |
| | PML | | | | 5 | | |
| Percutaneous absorption | | 33.27 | 41.04 | 37.25 | 32.13 | 98.52 | 87.41 |
| (μg/cm²/24 h) | | | | | | | |
| Patch | Initial | Colorless | Colorless | Colorless | Colorless | Colorless | Colorless |
| appearance | | transparent | transparent | transparent | transparent | transparent | transparent |
| | After 1 | Colorless | Colorless | Colorless | Colorless | Colorless | Colorless |
| | month | transparent | transparent | transparent | transparent | transparent | transparent |

| | | | | | | |
|---|---|---|---|---|---|---|
| Physical properties and | Good | Good | Good | Good | Good | Good |
| adhesive strength | | | | | | |
| Crystal formation | None | None | None | None | None | None |
| (after 1 month) | | | | | | |
| (microscopic observation) | | | | | | |

LNA: linoleic acid;
MYA: myristic acid;
OLA: oleic acid;
PGML: propylene glycol monolaurate

As shown in Table 5 above, the manufactured patches easily achieved percutaneous absorption when the C₈₋₁₈ aliphatic derivative was used as the percutaneous absorption enhancer, like the case of Examples 23 to 28.

### INDUSTRIAL APPLICABILITY

The formulation for percutaneous absorption of melatonin according to the present invention enables controlled drug release, has high stability, convenient to store and use, and is easy to manufacture. In addition, the percutaneous absorption formulation according to the present invention can significantly improve patient's medication compliance compared to an oral formulation, and at the same time, it can deliver an effective amount of a necessary drug to a patient, indicating that it can be advantageously used for the treatment of insomnia and sleep disorders.

1. A percutaneous absorption formulation comprising:
(a) a drug-containing adhesive layer which contains melatonin or a pharmaceutically acceptable salt thereof as an active ingredient, and a polymeric adhesive agent;
(b) a support layer; and
(c) a release layer.

2. The percutaneous absorption formulation of claim 1, wherein the drug-containing adhesive layer further contains a solubilizing agent, a crystallization inhibitor, a percutaneous absorption enhancer, and an antioxidant.

3. The percutaneous absorption formulation of claim 2, wherein the drug-containing adhesive layer contains, based on the total weight of the drug-containing adhesive layer, 3.0 to 20 wt % of melatonin or the pharmaceutically acceptable salt thereof, 1 to 30 wt % of the solubilizing agent, 0.05 to 5 wt % of the crystallization inhibitor, 1 to 30 wt % of the percutaneous absorption enhancer, 0.1 to 5 wt % of the antioxidant, and 50 to 94.85 wt % of a polymeric adhesive agent.

4. The percutaneous absorption formulation of claim 2, wherein the solubilizing agent is one or two or more selected from the group consisting of N-methylpyrrolidone, dipropylene glycol, propylene glycol, propylene carbonate, ethoxydiglycol, diethylene glycol monoethyl ether, triacetin, triethyl citrate, triethanolamine, tromethamine, bis-Tris, aminomethyl propanediol, aminoethyl propanediol, polyoxyethylene sorbitan monooleate, and PEG-8 caprylic/capric glycerides.

5. The percutaneous absorption formulation of claim 2, wherein the crystallization inhibitor for inhibiting crystal formation of the drug is one or two or more selected from the group consisting of polyvinylpyrrolidone, a methacrylic copolymer, an amino acrylic methacrylate copolymer, a butyl methacrylic methacrylate copolymer, and hydroxypropyl cellulose.

6. The percutaneous absorption formulation of claim 2, wherein the percutaneous absorption enhancer is a C₈₋₁₈ aliphatic derivative.

7. The percutaneous absorption formulation of claim 6, wherein the percutaneous absorption enhancer is one or two or more selected from the group consisting of linoleic acid, oleic acid, myristic acid, sorbitan monooleate, and propylene glycol monolaurate.

8. The percutaneous absorption formulation of claim 2, wherein the percutaneous absorption enhancer is one or two or more selected from the group consisting of glycerol lauryl alcohol, oleyl alcohol, isopropyl myristate, sorbitan monooleate, propylene glycol monolaurate, propylene glycol monooleate, oleoyl macrogolglycerides, oleic acid, lauroyl macrogol glyceride, linoleoyl macrogol glyceride, propylene glycol dicaprylate/caprate, sorbitan monostearate, sorbitan monooleate, glycerol monooleate, glycerol monolaurate, propylene glycol monolaurate, propylene glycol monocaprylate, sorbitan monolaurate, lauryl lactate, PEG-8 caprylic/capric triglycerides, polyoxyethylene sorbitan monolaurate, corn oil PEG-8 esters, and corn oil PEG-6 esters.

9. The percutaneous absorption formulation of claim 2, wherein the antioxidant is one or two or more selected from the group consisting of butyl hydroxy toluene, butyl hydroxy anisole, propyl galate, ascorbic acid, tocopherol, tocopherol acetate, and ascorbyl palmitate.

10. The percutaneous absorption formulation of claim 1, wherein the polymeric adhesive agent is an acrylic adhesive.

11. The percutaneous absorption formulation of claim 1, wherein the polymeric adhesive agent is an acrylic polymer adhesive agent composed of either acrylate or a copolymer of acrylate and vinyl acetate, wherein the acrylic polymer adhesive agent is one or two or more selected from the group consisting of (i) one having no functional group, (ii) one having a hydroxyl (—OH) group as a functional group, (iii) one having a carboxyl (—COOH) group as a functional group, and (iv) one having both a hydroxyl group and a carboxyl group as functional groups.

12. The percutaneous absorption formulation of claim 1, wherein the polymeric adhesive agent is an acrylic polymer adhesive agent composed of either acrylate or a copolymer of acrylate and vinyl acetate, wherein the acrylic polymer adhesive agent is (i) one having a carboxyl (—COOH) group as a functional group, or (ii) one having both a hydroxyl group and a carboxyl group as functional groups.

13. The percutaneous absorption formulation of claim 1, wherein the polymeric adhesive agent is an adhesive agent which comprises a hydrophobic polymer, wherein the hydrophobic polymer is one or two or more selected from the group consisting of polyisoprene, polyisobutylene, polybutadiene, polystyrene-butadiene copolymer, polystyrene-isoprene copolymer, styrene-isoprene-styrene block copolymer, styrene-butadiene-styrene block copolymer, butyl rubber, natural rubber, ethylene-vinyl acetate copolymer, polysiloxane, and methacrylic acid-based polymers.

14. The percutaneous absorption formulation of claim 13, wherein the polymeric adhesive agent further comprises a tackifying resin and a plasticizer, and comprises 20 to 60 wt % of the hydrophobic polymer, 20 to 50 wt % of the tackifying resin, and 2 to 30 wt % of the plasticizer.

15. The percutaneous absorption formulation of claim 1, wherein the support layer is one selected from the group consisting of a polyethylene terephthalate (PET) film, a polyethylene (PE) film, a polypropylene (PP) film, an ethylene vinyl acetate (EVA) film, a nylon film, a nonwoven fabric/PET laminate, a PET/PE laminate, and a PET/EVA laminate.

16. The percutaneous absorption formulation of claim 1, wherein the release layer is a release layer obtained by surface-treating one selected from the group consisting of a polyester film, a polyvinyl chloride film, a polyvinylidene chloride film, a polyethylene terephthalate (PET) film, a polyethylene (PE) film, a polyethylene/paper laminate, a PET/PE laminate, and a PET/EVA laminate, the surface-treating one being treated with a silicone or fluorine treatment agent.
